(19) United States
(12) Patent Application Publication
(10) Pub. No.: US 2013/0304176 A1
(43) Pub. Date: Nov. 14, 2013

(54) INTEGRATED BANDAGE AND ELECTRICAL STIMULATION TRANSCUTANEOUS ELECTRICAL NEURON-STIMULATION (TENS) DEVICE

(71) Applicants: Kent Riddle, Greensboro, NC (US); Barry Cantrell, Greensboro, NC (US)

(72) Inventors: Kent Riddle, Greensboro, NC (US); Barry Cantrell, Greensboro, NC (US)

(21) Appl. No.: 13/886,871

(22) Filed: May 3, 2013

Related U.S. Application Data
(60) Provisional application No. 61/642,809, filed on May 4, 2012.

(51) Int. Cl. A61N 1/04

(52) U.S. Cl. CPC A61N 1/0456
USPC 607/149

(57) ABSTRACT

A bandage with an integrated, wireless, transcutaneous electrical neuron-stimulation (TENS) device, and method of using the same. The integrated bandage and TENS device provides electric stimulation to an anatomical site that is covered by the bandage. The bandage includes controls that enable the user to control the intensity levels of the electric stimulation, which is provided through at least one electrode. The electrical circuit in the TENS device provides biphasic or monophasic sequence of pulses to the electrodes, and the sequence of pulses form a plurality of waveforms available for specific clinical needs.

This application claims the benefit of U.S. Provisional Application No. 61/642,809, filed May 4, 2012, the disclosure of which is expressly incorporated herein by reference.

This patent application is related to U.S. patent application Ser. No. 11/434,453, entitled “Combination electrode-battery and, programming assembly for a miniature wireless transcutaneous electrical neuro or muscular-stimulation unit,” filed May 15, 2006; U.S. patent application Ser. No. 10/208,223, entitled “Combination electrode-battery assembly for a miniature wireless transcutaneous-electrical neuro or muscular-stimulation unit,” filed Jul. 30, 2002; U.S. patent application Ser. No. 09/853,440, now U.S. Pat. No. 6,607,500 entitled “Integrated Cast and Muscle Simulation,” filed May 10, 2001; U.S. patent application Ser. No. 09/854,297, entitled “Two Part TENS Bandage,” filed May 10, 2001; and U.S. patent application Ser. No. 09/350,426, now U.S. Pat. No. 6,445,955, entitled “Miniature Wireless Transcutaneous Electrical Neuro or Muscular-Stimulation Unit,” filed Jul. 8, 1999; the entirety of which are hereby incorporated by reference.

### BACKGROUND OF THE INVENTION

The present invention generally relates to an integrated bandage and electrical stimulation transcutaneous electrical neuron-stimulation (TENS) device, and more specifically relates to a treatment device that may provide electric stimulation to an anatomical site that is covered by a bandage.

### SUMMARY OF THE INVENTION

Embodiments of the present invention provide devices and methods for TENS bandages. The device may be a disposable bandage designed to be sold directly to consumers, without a physician's prescription, for the temporary relief of pain. The device may adhere to a person's skin, and emit controlled and focused electrical signals. The device may also emit fixed or modulated electrical signals through a grid of electrodes.

In one embodiment, the device comprises a bandage and a treatment electrode coupled to the bandage. In another embodiment, the device comprises an electrical circuit, a power source, a flexible electrical grid, an adhesive surface, controls (e.g., on/off, signal type, signal strength), and a visual indicator in a single, integrated, disposable package. In one embodiment, the device may be configured to operate for up to eight hours, without any rechargeable, replaceable, or interchangeable parts.

### BRIEF DESCRIPTION OF THE DRAWINGS

The foregoing, as well as other objects of the present invention, will be further apparent from the following detailed description of the preferred embodiment of the invention, when taken together with the accompanying specification and the drawings, in which:

FIG. 1 shows a device according to a first embodiment of the present invention.

FIG. 2 shows a device according to a second embodiment of the present invention.

### DESCRIPTION OF THE PREFERRED EMBODIMENTS

The accompanying drawings and the description which follows set forth this invention in its preferred embodiments. However, it is contemplated that persons generally familiar with this art will be able to apply the novel characteristics of the structures illustrated and described herein in other contexts by modification of certain details. Accordingly, the drawings and description are not to be taken as restrictive on the scope of this invention, but are to be understood as broad and general teachings.

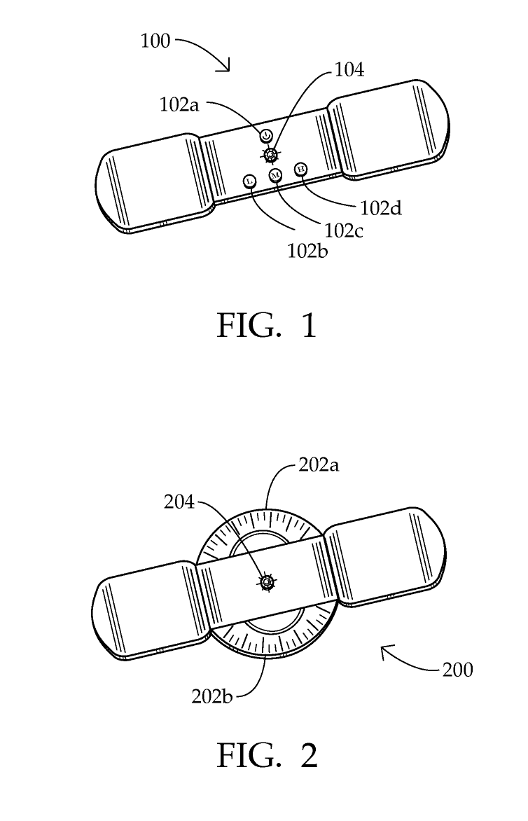

In one illustrative embodiment of the present invention as shown in FIG. 1, a device 100 comprises a bandage. In the embodiment shown in FIG. 1, the bandage has a width of approximately two inches, and a length of approximately six inches. Embodiments of the device are not limited to this size. Other embodiments of the device may be shorter, longer, thinner, or wider.

In one embodiment, the device is configured for adhesion and use on various body parts. In another embodiment, the device is configured for use in a specific location of the body, such as the lower back. The device may be designed to appear inconspicuous, for example, to blend in with the surrounding skin. In one variation, the device may appear similar in size and color to ordinary, generic adhesive bandages.

Device 100 may relieve, reduce, or eliminate pain through the emission of controlled and focused electrical signals. In one variation, the device may be configured to produce a continuous perception of electrical paresthesia, or tingling, without muscle contraction. The emission of controlled, focused electrical signals may provide temporary or permanent relief of pain.

In the embodiment shown in FIG. 1, the device comprises user controls 102a, 102b, 102c, and 102d. User controls 102 may comprise large, easy to read buttons. The user controls 102 may be mounted on the surface of device 100, and require a low amount of pressure for operation. 102a comprises a power button, to turn the device on and off. 102b, 102c, and 102d comprise level buttons, corresponding to a low level (“L”), a medium level (“M”), and a high level (“H”).

The device may comprise other user controls (not shown). For example, a device may comprise higher and lower level controls. As shown in the embodiment illustrated in FIG. 2, the device 200 may also be configured with a dial (202a, 202b) that allows the user to adjust the level of the emitted electrical signal by rotating the dial (202a, 202b).

In the embodiments shown in FIGS. 1 and 2, the device 100, 200 comprises a visual indicator (104, 204, respectively). For example, the device may comprise a light, such as a light emitting diode (LED). The visual indicator 104, 204 may indicate whether the device is in use. For example, the visual indicator may be illuminated while electrical signals are being emitted from the device. The visual indicator may be configured to blink at a regular interval while the device is on.

In one example, a light may immediately come on when the device is turned on. The light may blink for one second intervals for 10 seconds. Then, after 10 seconds, the light may fade to black, and blink once every 45 seconds while the device is turned on. If the device user modifies the function of the device, for example, by adjusting the device's intensity, the light timer may be reset. When the device timer is reset, it may repeat the initial start-up routine, and blink once every second for ten seconds. The visual indicator may also provide other statuses. For example, the visual indicator may remain solidly on to indicate that battery life is low.

The device may comprise an internal power supply (not shown in FIG. 1 or 2). The power supply may comprise a battery, configured to operate for eight hours, and be disposed upon depletion. The power supply may be integrated into the device without any replaceable parts. The power supply may be positioned in the middle of the bandage.

The device may also comprise an electronics module (not shown in FIG. 1 or 2). The power supply may supply power to an electronics module. The electronics module may be located inside a middle portion of the bandage. The electronics module may comprise an electrical circuit which provides a biphasic or monophasic sequence of pulses to the electrodes, wherein the sequence of pulses form a plurality of pre-programmable waveforms available for specific clinical needs.

The device may comprise one or more electrodes configured to emit an electrical signal. In one variation, the electrical signal is a fixed electrical signal. In another variation the electrical signal is a modulated signal. A positive electrode and a negative electrode may be positioned on either side of a power supply and/or electronic module.

The electrical signal may be emitted through a grid of integrated electrodes. In one embodiment, the grid comprises a pair of 2 inch by 2 inch electrodes positioned at opposite ends of the device. The electrode grid may be configured to provide maximum signal transmission over a two inch by two inch area at each end of the device.

In an alternate form, the plurality of electrodes have varying shapes and sizes and can be affixed directly to a site or other area requiring electrical neuro or muscular-stimulation anywhere on the user's body, the electrodes are positioned at a specified distance from the electronics module wherein the electrodes are swivel or fixed electrodes which allow for optimal placement of the electrodes at the pain site or the area requiring electrical neuro or muscular-stimulation.

The circuit may be current limiting, or have a constant current, to avoid over-stimulation due to variation of skin impedance. The polarity of the electrical signals may be reversed every second.

The pulse rate of the electric signals may be fixed at a medium setting, such as 85 Hz, with the pulse set to a low setting such as 75 microseconds, and an adjustable intensity ranging from 0 milliamps to 30 milliamps. In one variation, the maximal intensity is higher than 30 milliamps.

In another variation, the device may be configured with an intensity range of 40 milliamps based on a 500 ohm load. The device may be configured with a 20 vole intensity range based on a 500 ohm load. The device may have 6 biphasic stimulation modes. The device may comprise 2 modes per electronic battery. The device may comprise 6 electronic battery strips.

In an alternate form, the plurality of waveforms are comprised of but not limited to, a conventional modulation low mode and a microcurrent mode wherein the conventional-low mode or the modulation-low mode supplies to the plurality of electrodes biphasic or monophasic pulses of about 0-30 milliamperes at a frequency of about 80-100 Hz having a pulse width of about 75 microseconds and the microcurrent mode supplies to the plurality of electrodes the biphasic pulse of about 0.3-100 Hz.

In an alternate embodiment, the plurality of waveforms are comprised of but not limited to, a conventional-low mode and a conventional-high mode, a modulation-low mode and a modulation-high mode, an acupuncture-like low mode and an acupuncture-like high mode, a microcurrent mode, burst mode and a cycling mode wherein the conventional-low mode supplies to the plurality of electrodes biphasic or monophasic pulses of about 0-60 milliamperes at a frequency of about 100 Hz having a pulse width of about 75 microseconds, the conventional high mode supplies to the electrodes biphasic or, monophasic pulses of about 0-100 milliamperes at a frequency of about 100 Hz having a pulse width of about 125 microseconds, the-modulation-low mode supplies to the electrodes biphasic or monophasic pulses of about 0-60 milliamperes at a frequency of about 50-100 Hz having a pulse width of about 75-100 microseconds, the modulation-high mode supplies to the electrodes biphasic or monophasic pulses of about 0-100 milliamperes at a frequency of about 75-100 Hz having a pulse width of about 100-125 microseconds, the acupuncture-like low mode supplies to the plurality of electrodes biphasic pulses of about 0-60 milliamperes at a frequency of about 1 Hz having a pulse width of about 75 microseconds, the acupuncture-like high mode supplies to the electrodes biphasic pulses of about 0-100 milliamperes at a frequency of about 2 Hz having a pulse width of about 125 microseconds, the microcurrent mode supplies to the plurality of electrodes biphasic pulse of about 0-100 microamperes at a frequency of about 0.3-100 Hz, the burst mode supplies to the plurality of electrodes biphasic or monophasic pulses of about 0-100 milliamperes at a frequency of about 100 Hz having a pulse width of about 75 microseconds for a duration of three seconds on and three seconds off, and the cycling mode allows the user to program two or more modes into an individualized program.

In an alternate form, the plurality of waveforms are comprised of but not limited to, three unique muscle stimulation modes, a conventional mode, a modulation mode and three alternative cycling modes, wherein the first muscle stimulation mode supplies to the plurality of electrodes biphasic or monophasic pulses typically of about 0-100 milliamperes at a frequency of about 45 Hz at a pulse width of about 300 microseconds for approximately 5 minutes on and 5 minutes off, wherein the second muscle stimulation mode supplies to the plurality of electrodes biphasic or monophasic pulses typically of about 0-100 milliamperes at a frequency of about 45 Hz at a pulse width of about 300 microseconds for approximately 10 minutes on and 10 minutes off, the third muscle stimulation mode supplies to the plurality of electrodes biphasic or monophasic pulses typically of about 0-100 milliamperes at a frequency of about 45 Hz at a pulse width of about 300 microseconds for approximately 10 minutes on and 50 minutes off, the conventional mode supplies to the plurality of electrodes biphasic or monophasic pulses typically of about 0-100 milliamperes at a frequency of about 125 Hz at a pulse width of about 125 microseconds, the modulation mode supplies to the plurality of electrodes biphasic or monophasic pulses typically of about 0-100 milliamperes at a frequency of about 75-100 Hz at a pulse width of about 100-125 microseconds the first alternative cycling mode supplies to the plurality of electrodes a sequence of biphasic or monophasic pulses comprised of the modulation mode for about 3 minutes followed by the first muscle stimulation mode for about 9 minutes, followed by the modulation for about 3 minutes, the second alternative cycling mode supplies to the plurality of electrodes a sequence of biphasic or monophasic pulses comprised of the modulation mode for about 3 minutes followed by the first muscle stimulation mode for about 9 minutes, followed by the modulation mode for about 3 minutes, and the third alternative cycling mode supplies to the plurality of electrodes a sequence of biphasic or monophasic pulses comprised of the modulation mode for about 3 minutes followed by the third muscle stimulation mode for about 9 minutes, followed by the modulation mode for about 3 minutes.

As referenced above, the device may be configured to emit multiple levels of electrical signals. In one variation, the device may be configured to emit a low, medium, and high intensity level. For example, the device may be configured to output 10 milliamps, 20 milliamps and 30 milliamps.

The device may be configured to delay or slowly adjust output intensity levels. For example, after a user advances the dial (202a, 202b) to a position associated with a higher level of electrical output, the device may increase to the next highest intensity level over a two-second “ramp-up” period. Such a ramp-up period may avoid or eliminate a sensation of sudden action. In other embodiments, the device may not comprise a ramp-up delay.

In one embodiment, the device adheres to a person's skin. For example, the device may comprise one or more adhesive pads. An adhesive pad may be affixed to the device at opposite ends to provide effective anchoring of the device on a person. The adhesive may be a non-allergenic adhesive. The adhesive may be configured to be re-used. In one variation, the device may be applied to a patient, removed from a patient, and re-applied to the patient. In one variation, the adhesive may be configured to adhere to the person's skin in moist, damp or water-logged conditions.

The device may be configured with certain safety features. For example, the entire device may be waterproof. As another example, the battery and/or electrodes may be encased in an impermeable casing. An over the counter device may be configured to operate within certain ranges specified by government authorities.

The device may comprise a contact probe. The contact probe may be configured to detect contact with a person's skin. In one embodiment, the device is configured to emit an electrical signal when the contact probe detects contact with a person's skin. In a variation, the device may emit an electrical signal after the contact probe detects continuous contact with a person's skin. For example, the device may emit an electrical signal after the contact probe detects continuous contact with a person's skin for two, three or more seconds. Such a delay may give a person time to effectively secure the device.

The device may comprise a flexible, cloth-like material. such as found in a bandage or an adhesive bandage. As examples, the material may comprise a woven fabric, plastic, or latex rubber.

In one embodiment, the device is configured to provide levels of stimulus below levels requiring a physician's prescription. Such embodiments may be made commercially available, or “over-the-counter” (OTC) without a physician's prescription. In one embodiment, an over-the-counter device may not output an electrical signal above 25 microcoulumbs. In another variation, the device may be configured to be operated by a professional. For example, a device configured to output an electrical signal above 25 microcoulumbs may be configured to be operated by a physician.

An OTC device may have a fixed or finite operational time. In one embodiment, the device is configured to operate for up to eight hours. In one embodiment an OTC device may have no replaceable, rechargeable, or interchangeable parts.

Additionally, the device's electrodes may be manufactured with a type of balm, such as menthol, or medication, such as ibuprofen gel, that is integrated into the electrode surfaces. This allows the device to also serve as a transdermal delivery system of either over the counter or prescribed medications.

The foregoing description of the embodiments, including preferred embodiments, of the invention has been presented only for the purpose of illustration and description and is not intended to be exhaustive or to limit the invention to the precise forms disclosed. Numerous modifications and adaptations thereof will be apparent to those skilled in the all without departing from the spirit and scope of this invention.

What is claimed is:

1. An apparatus comprising:
a bandage; and
a wireless transcutaneous electrical neuron-stimulation (TENS) device integrated with the bandage for providing electric stimulation to anatomical site that is covered by the bandage.

2. The apparatus of claim 1 wherein the apparatus comprises at least one integrated control for controlling the TENS device.

3. The apparatus of claim 2 wherein the at least one integrated control comprises at least one button for controlling operations selected from the group consisting of power on, power off, and electric stimulation intensity.

4. The apparatus of claim 2 wherein the at least one integrated control comprises a dial for controlling electric stimulation intensity.

5. The apparatus of claim 2 wherein an intensity level of the electric stimulation gradually changes in response to activation of the at least one integrated control.

6. The apparatus of claim 1 further comprising a visual indicator that visually indicates status information selected from the group consisting of power on, power off, and electric stimulation intensity.

7. The apparatus of claim 1 further comprising an integrated power supply the supplies power to the TENS device.

8. The apparatus of claim 7 wherein the integrated power supply is a battery.

9. The apparatus of claim 7 wherein integrated power supply is configured to not be replaceable or interchangeable within the apparatus.

10. The apparatus of claim 1 wherein the TENS device comprises at least one electrode for emitting an electrical signal as the electric stimulation.

11. The apparatus of claim 10 wherein the electric signal is selected from the group consisting of a fixed signal and a modulated signal.

12. The apparatus of claim 10 further comprising an integrated power supply, wherein the integrated power supply and the at least one electrode are encased in a casing.

13. The apparatus of claim 10 wherein the at least one electrode further comprises a balm that is configured for transdermal contact with the anatomical site, wherein the balm is selected from the group consisting of menthol, gel, and medication.

14. The apparatus of claim 1 further comprising a grid of electrodes for emitting an electrical signal as the electric stimulation.

15. The apparatus of claim 1 wherein the TENS device is configured to emit varying levels of electric stimulation ranging up to about 30 milliamps.

16. The apparatus of claim 1 wherein the TENS device is configured to emit varying levels of electric stimulation ranging above about 30 milliamps.

17. The apparatus of claim 1, wherein the TENS device is configured to emit electrical stimulation at a level not exceeding about 25 microcoulombs.

18. The apparatus of claim 10 wherein the TENS device further comprises an electrical circuit that provides a biphasic or monophasic sequence of pulses to the at least one electrode, wherein the sequence of pulses forms a plurality of waveforms available for specific clinical needs.

19. The apparatus of claim 18 wherein the plurality of waveforms comprise waveforms selected from the group consisting of:
(i) conventional low mode wherein the at least one electrode is supplied with biphasic or monophasic pulses of about 0 to about 60 milliamperes at a frequency of about 100 Hz having a pulse width of about 75 microseconds;
(ii) conventional high mode wherein the at least one electrode is supplied with biphasic or monophasic pulses of about 0 to about 100 milliamperes at a frequency of about 100 Hz having a pulse width of about 125 microseconds;
(iii) modulation-low mode wherein the at least one electrode is supplied with biphasic or monophasic pulses of about 0 to about 60 milliamperes at a frequency of about 50 to about 100 Hz having a pulse width of about 75 to about 100 microseconds;
(iv) modulation-high mode wherein the at least one electrode is supplied with biphasic or monophasic pulses of about 0 to about 100 milliamperes at a frequency of about 75 to about 100 Hz having a pulse width of about 100 to about 125 microseconds;
(v) acupuncture-like low mode wherein the at least one electrode is supplied with biphasic pulses of about 0 to about 60 milliamperes at a frequency of about 1 Hz having a pulse width of about 75 microseconds;
(vi) acupuncture-like high mode wherein the at least one electrode is supplied with biphasic pulses of about 0 to about 100 milliamperes at a frequency of about 2 Hz having a pulse width of about 125 microseconds;
(vii) microcurrent mode wherein the at least one electrode is supplied with biphasic pulse of about 0 to about 100 microamperes at a frequency of about 0.3 to about 100 Hz; and
(viii) burst mode wherein the at least one electrode is supplied with biphasic or monophasic pulses of about 0 to about 100 milliamperes at a frequency of about 100 Hz having a pulse width of about 75 microseconds for a duration of about three seconds on and about three seconds off.

20. The apparatus of claim 19 further comprising a cycling mode wherein the TENS device cycles between at least two of the conventional-low mode, conventional-high mode, modulation-low mode, modulation-high mode, acupuncture-like low mode, acupuncture-like high mode, microcurrent mode, and burst modes.

21. The apparatus of claim 1 further comprising a contact probe for detecting contact a person's skin, wherein the TENS device is configured to emit the electrical stimulation when the contact probe detects contact with the person's skin.

22. The apparatus of claim 1 wherein the TENS device is configured to emit electrical stimulation at levels not exceeding that which would require a physician's prescription.

23. The apparatus of claim 1 wherein the bandage is formed of material selected from the group consisting of woven fabric, plastic, and latex rubber.

24. The apparatus of claims 1 wherein the apparatus is waterproof.

25. The apparatus of claim 1 wherein the bandage is self-adhesive.

26. The apparatus of claim 1 wherein the bandage is reusable.

27. A method of using a bandage with an integrated transcutaneous electrical neuron-stimulation (TENS) device for providing electric stimulation to anatomical site that is covered by the bandage, said method comprising the steps of:
(i) applying the bandage to an anatomical site on a human body; and
(ii) manipulating at least one control on the bandage for initiating and controlling an intensity of the electric stimulation intensity provided by the TENS device to the anatomical site.
